(19) United States
(12) Patent Application Publication
(10) Pub. No.: US 2017/0164884 A1
(43) Pub. Date: Jun. 15, 2017

(54) MEASURING RESPIRATION RATE WITH MULTI-BAND PLETHYSMOGRAPHY

(71) Applicant: Apple Inc., Cupertino, CA (US)

(72) Inventors: Daniel J. CULBERT, Los Gatos, CA (US); Ian R. SHAPIRO, Saratoga, CA (US)

(21) Appl. No.: 15/039,392

(22) PCT Filed: Dec. 30, 2013
(86) PCT No.: PCT/US2013/078385

§ 371 Date: May 25, 2016

(51) Int. Cl. A61B 5/20; A61B 5/024; A61B 5/087

(52) U.S. Cl. CPC A61B 5/205; A61B 5/0873; A61B 5/02427

(57) ABSTRACT

A photoplethysmogram (PPG) signal may be obtained from a pulse oximeter, which employs a light emitter and a light sensor to measure the perfusion of blood to the skin of a user, and multiple wavelengths of light may be employed. For various wavelengths, relatively long wavelengths may interrogate relatively deep blood vessels in comparison to relatively short wavelengths, which may interrogate relatively shallow blood vessels. Accordingly, for co-located emitters of different wavelengths, there may be a time delay in the pulse signal measured by each wavelength. The time delay as a function of time may vary according to the constriction and dilation of the blood vessels, which itself may vary according to the respiratory rate of a user.

### FIELD OF THE DISCLOSURE

This relates generally to processing of a photoplethysmogram (PPG) signal.

### BACKGROUND OF THE DISCLOSURE

A photoplethysmogram (PPG) signal may be obtained from a pulse oximeter, which employs a light emitter and a light sensor to measure the perfusion of blood to the skin of a user. However, the signal may be compromised by noise due to motion artifacts. That is, movement of the body of a user may cause the skin and vasculature to expand and contract, introducing noise to the signal.

### SUMMARY OF THE DISCLOSURE

A photoplethysmogram (PPG) signal may be obtained from a pulse oximeter, which employs a light emitter and a light sensor to measure the perfusion of blood to the skin of a user. However, the signal may be compromised by noise due to motion artifacts. That is, movement of the body of a user may cause the skin and vasculature to expand and contract, introducing noise to the signal. To address the presence of motion artifacts, examples of the present disclosure can receive light information from two light sensors situated in a line parallel to the direction of the blood pulse wave. The light information from each sensor may include the same noise signal, and thus subtracting one from the other can result in a heart rate signal where the noise has been canceled out. In some examples, a signal from one of the light sensors may be multiplied by a scaling factor before cancellation to account for response differences in each light sensor.

Further, multiple wavelengths of light may be employed. For various wavelengths, relatively long wavelengths may interrogate relatively deep blood vessels in comparison to relatively short wavelengths, which may interrogate relatively shallow blood vessels. Accordingly, for co-located emitters of different wavelengths, there may be a time delay in the pulse signal measured by each wavelength. For example, green light may interrogate relatively shallow blood vessels near the surface of the skin, and red light may interrogate relatively deep blood vessels deeper beneath the skin, and thus the pulse signal measured by the green light may be delayed in time compared to the pulse signal measured by the red light. The time delay as a function of time may vary according to the constriction and dilation of the blood vessels, which itself may vary according to the respiratory rate of a user. In this way, the light information of various wavelengths may be used to compute such a time delay as a function of time, and a respiratory rate signal may be determined accordingly.

### BRIEF DESCRIPTION OF THE DRAWINGS

FIG. 1 illustrates an electronic device having light sensors for determining a heart rate signal according to examples of the disclosure.

FIG. 2 illustrates a method of computing a heart rate signal wherein noise due to motion artifacts has been canceled according to examples of the disclosure.

FIG. 3 illustrates a logical block diagram of a process of canceling noise to compute a heart rate signal according to examples of the disclosure.

FIG. 4 illustrates an electronic device having a light sensor for determining a respiratory rate signal according to examples of the disclosure.

FIG. 5 illustrates a method of computing a physiological signal corresponding to a respiratory rate according to examples of the disclosure.

FIG. 6 is a block diagram illustrating an exemplary API architecture, which may be used in some examples of the disclosure.

FIG. 7 illustrates an exemplary software stack of an API according to examples of the disclosure.

FIG. 8 is a block diagram illustrating exemplary interactions between the touch screen and other components of the device according to examples of the disclosure.

FIG. 9 is a block diagram illustrating an example of a system architecture that may be embodied within any portable or non-portable device according to examples of the disclosure.

### DETAILED DESCRIPTION

In the following description of examples, reference is made to the accompanying drawings which form a part hereof, and in which it is shown by way of illustration specific examples that can be practiced. It is to be understood that other examples can be used and structural changes can be made without departing from the scope of the disclosed examples.

A photoplethysmogram (PPG) signal may be obtained from a pulse oximeter, which employs a light emitter and a light sensor to measure the perfusion of blood to the skin of a user. However, the signal may be compromised by noise due to motion artifacts. That is, movement of the body of a user may cause the skin and vasculature to expand and contract, introducing noise to the signal. To address the presence of motion artifacts, examples of the present disclosure can receive light information from two light sensors situated in a line parallel to the direction of the blood pulse wave. The light information from each sensor may include the same noise signal, and thus subtracting one from the other can result in a heart rate signal where the noise has been canceled out. In some examples, a signal from one of the light sensors may be multiplied by a scaling factor before cancellation to account for response differences in each light sensor.

Further, multiple wavelengths of light may be employed. For various wavelengths, relatively long wavelengths may interrogate relatively deep blood vessels in comparison to relatively short wavelengths, which may interrogate relatively shallow blood vessels. Accordingly, for co-located emitters of different wavelengths, there may be a time delay in the pulse signal measured by each wavelength. For example, green light may interrogate relatively shallow blood vessels near the surface of the skin, and red light may interrogate relatively deep blood vessels deeper beneath the skin, and thus the pulse signal measured by the green light may be delayed in time compared to the pulse signal measured by the red light. The time delay as a function of time may vary according to the constriction and dilation of the blood vessels, which itself may vary according to the respiratory rate of a user. In this way, the light information of various wavelengths may be used to compute such a time delay as a function of time, and a respiratory rate signal may be determined accordingly.

Although examples disclosed herein may be described and illustrated herein primarily in terms of one or two sensors and two emitters, it should be understood that the examples are not so limited, but are additionally applicable to devices including any number of sensors and emitters.

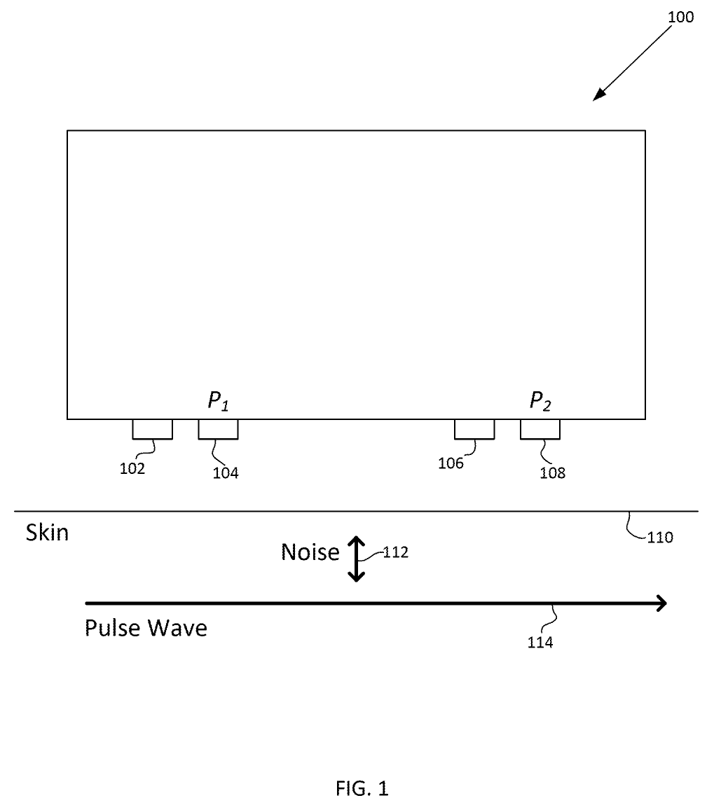

FIG. 1 illustrates an electronic device 100 having light sensors for determining a heart rate signal according to examples of the disclosure. A first light sensor 104 (P₁) may be co-located with a first light emitter 102 on a surface of the electronic device 100. Additionally, a second light sensor 108 (P₂) may be co-located with a second light emitter 108 on a surface of the electronic device 100. The electronic device 100 may be situated such that the sensors 104 and 108 and the emitters 102 and 106 are proximate the skin 110 of a user, so that light from a light emitter can be incident on the skin. For example, the electronic device 100 may be held in a user's hand or strapped to a user's wrist, among other possibilities.

A portion of the light from a light emitter may be absorbed by the skin, vasculature, and/or blood, among other possibilities, and a portion may be reflected back to a light sensor co-located with the light emitter. The light sensors 104 and 108 may be situated in a line parallel to a blood pulse wave 114, and the signal from each sensor can include a heart rate signal s due to the pulse wave. Due to a distance between the two sensors along the direction of the pulse wave 114, the signal from the first sensor may include the heart rate signal s(t), whereas the signal from the second sensor may include the time-shifted signal s(t+δ), where δ depends on the distance between the sensors and the blood pulse wave velocity.

Additionally, the signal from each sensor may include noise 112 (N) due to motion artifacts from the expansion and contraction of skin, vasculature, and other parts of the body as a user moves. Such noise can be modeled as a planar wave perpendicular to the direction of the pulse wave. The sensors 104 and 108 can be situated close enough together such that the component of noise affects each signal in the same way. That is, the signal from each sensor 104 and 108 can include the same noise factor N(t) with minimal perceived time-shifting of the noise signal between the two sensors.

Additionally, in some examples, due to response variations among light sensors, the signals from sensors 104 and 108 may include a factor k₁ and k₂, respectively. Accordingly, the signals at the sensors may be modeled by equations (1) and (2):

P₁(t)=k₁(s(t)+N(t)) (1)

P₂(t)=k₂(s(t+δ)+N(t)) (2)

Based on this model, the noise N(t) can be canceled from the signal by multiplying the signal P₂(t) by a scaling factor K, a ratio of the factors k₁ and k₂, and subtracting from the signal P₁(t), as shown in equations (3) and (4):

K=k₁/k₂ (3)

X(t)=P₁(t)−KP₂(t)=k₁(s(t)−s(t+δ)) (4)

The resulting signal X(t) can approximate a heart rate signal as a scalar multiple of a differential of the actual heart rate signal s.

In some examples, the electronic device may be calibrated to determine a proper scaling factor K. For example, a scaling factor K may be chosen such that the correlation between the signal X(t) and a template heart rate PPG signal is maximized. In some examples, independent component analysis methods may be used to select a scaling factor K that separates the signal X(t) as independent from signal N(t), each as components in the signal P₁(t). Other known methods of selecting and/or optimizing for a scaling factor K may be used.

In some examples, the distance between the sensors 104 and 108 may be chosen such that (1) the pulse wave transit time δ between the two sensors is large enough so that the heart rate signals s(t) and s(t+δ) do not cancel out, and (2) the sensors are close enough that the noise due to motion artifacts affects each signal in the same way at the same time (i.e., each signal has the same N(t)).

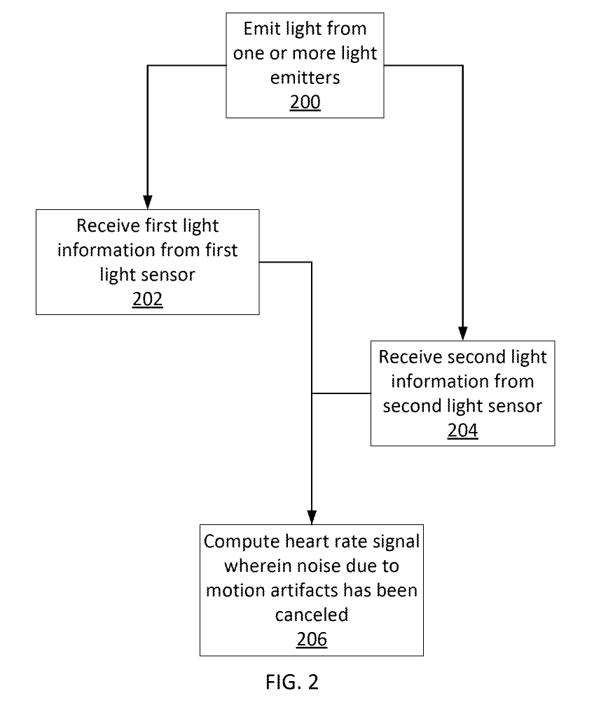

FIG. 2 illustrates a method of computing a heart rate signal wherein noise due to motion artifacts has been canceled according to examples of the disclosure. Light may be emitted from one or more light emitters (200). As illustrated in FIG. 1, a plurality of light emitters may be on a surface of an electronic device, each light emitter co-located with a corresponding light sensor. First light information may be received from a first light sensor (202), and second light information may be received from a second light sensor (204). In some examples, a light emitter may emit light, the light may travel to the skin of a user, and a portion of the light may reflect to the co-located light sensor. Accordingly, the first light information may indicate an amount of light from a first light emitter that has been reflected by the skin, blood, and/or vasculature of the user, among other possibilities. In some examples, the first light information may indicate an amount of light from the first light emitter that has been absorbed by the skin, blood, and/or vasculature of the user.

The light emitters may produce light in ranges corresponding to infrared (IR), green, amber, blue, and/or red light, among other possibilities. Additionally, the light sensors may be configured to sense light having certain wavelengths more powerfully than light having other wavelengths. For example, if the first light emitter emits light having a wavelength in the IR range, then the first light sensor may be configured to sense light in the IR range more powerfully than light in the green range. That is, the incidence of light in the IR range may produce a stronger response in the first light sensor than the incidence of light in the green range. In this way, the first light sensor can be configured so as to sense the light produced by the first light emitter more powerfully than the light produced by the second light emitter, for example. In some examples, each light emitter may produce light in the same wavelength.

In some examples, a light emitter may be a light emitting diode (LED) and a light sensor may be a photodiode. The light information may include information produced by the photodiode. For example, the light information may include a voltage reading that corresponds to light absorbed by the photodiode. In some examples, the light information may include some transformation of raw signal produced by the photodiode, such as through filtering, scaling, or other signal processing.

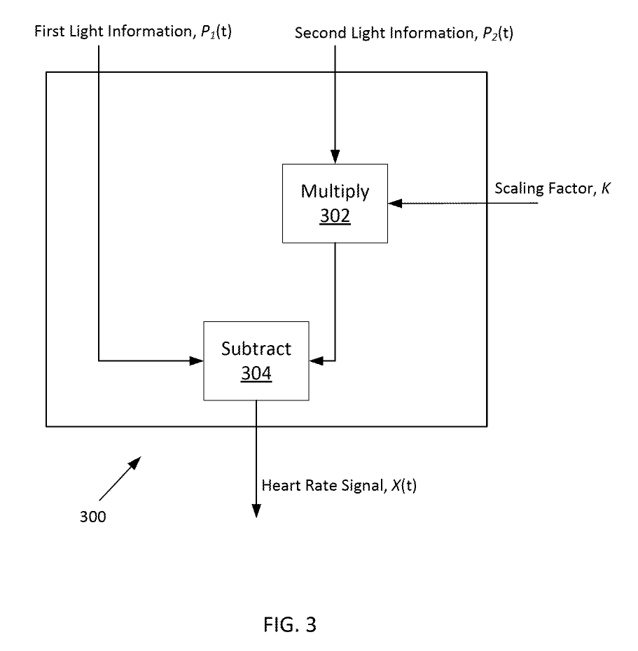

Based on the first and second light information, a heart rate signal may be computed, wherein noise due to motion artifacts has been canceled (206). For example, the second light information may be multiplied by a scaling factor and subtracted from the first light information to obtain the heart rate signal, as discussed above with reference to equations (1)-(4) and illustrated in FIG. 3. In some examples, receiving the second light information may include receiving the second light information already scaled, and thus computing the heart rate signal may involve merely subtracting the second light information from the first light information. In some examples, the heart rate signal itself may multiplied by a scaling factor.

FIG. 3 illustrates a logical block diagram of a process of canceling noise to compute a heart rate signal according to examples of the disclosure. In some examples, module 300 and functions 302 and 304 may each be implemented in software or hardware. Second light information P₂(t) may be multiplied by a scaling factor K at function 302. The output of function 302 may then be subtracted from the first light information P₁(t) at function 304. The output of function 304 may be the heart rate signal X(t).

In some examples, multiple wavelengths of light may be employed to produce pulse signals having a time delay. For various wavelengths, relatively long wavelengths may interrogate relatively deep blood vessels in comparison to relatively short wavelengths, which may interrogate relatively shallow blood vessels. Accordingly, for co-located light emitters of different wavelengths, there may be a time delay in the pulse signal measured by relatively short wavelengths. This time delay can be used to cancel motion artifacts as described above with reference to sensors at different locations. The following equations (5) and (6) describe a model utilizing multiple wavelengths (e.g., infrared (IR) and green):

P_{IR}(t)=k₁(s(t)+N(t)) (5)

P_{Green}(t)=k₂(s(t+Δ+ø(t))+N(t)) (6)

In equations (5) and (6), Δ may be a measurable, and invariant lag between IR and green light, ø(t) may be a periodic modulation of this lag. Once ø(t) is estimated, as described below, the same principles apply as in equations (1) through (4), and the heart rate signal can be computed with motion artifacts having been canceled.

As discussed above, for co-located light emitters of different wavelengths, there may be a time delay in the pulse signal measured by each wavelength. For example, green light may interrogate relatively shallow blood vessels near the surface of the skin, and red light may interrogate relatively deep blood vessels deeper beneath the skin, and thus the pulse signal measured by the green light may be delayed in time compared to the pulse signal measured by the red light. The time delay as a function of time may vary according to the constriction and dilation of the blood vessels, which itself may vary according to the respiratory rate of a user. In this way, the light information of various wavelengths may be used to compute such a time delay as a function of time, and a respiratory rate signal may be determined accordingly.

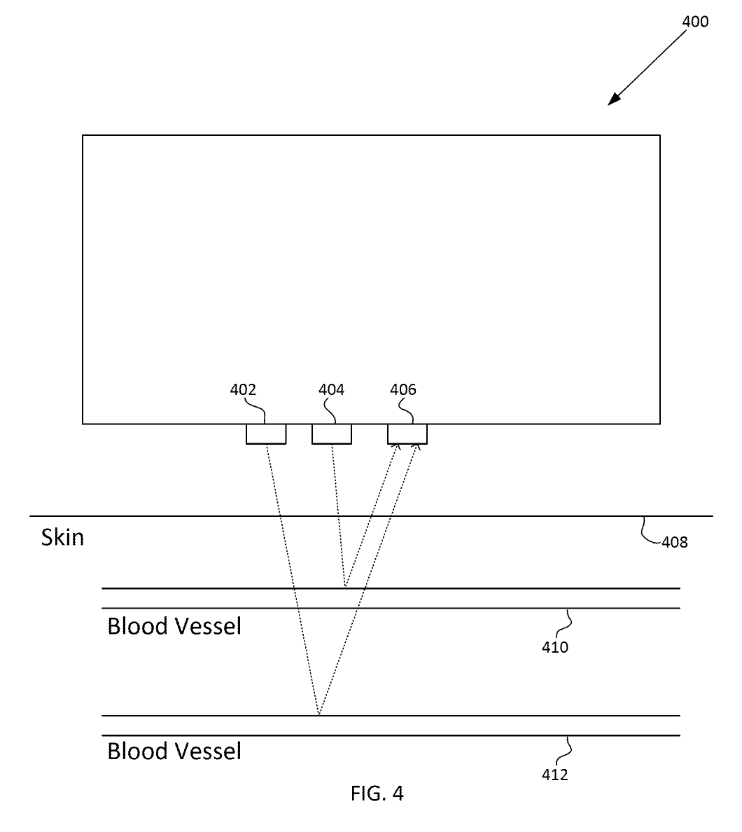

FIG. 4 illustrates an electronic device having a light sensor for determining a respiratory rate signal according to examples of the disclosure. An electronic device 400 may have a plurality of light emitters 402 and 404, and a light sensor 406. Light may be emitted from the plurality of emitters, and a portion of the light may be incident on one or more physiological systems, including the skin 408 and one or more blood vessels 410 and 412, among many other possibilities. Relatively long wavelengths may penetrate to deeper blood vessels than relatively short wavelengths. FIG. 4 illustrates a first emitter 402 emitting light having a first wavelength (e.g., in the infrared (IR) range) and a second emitter 404 emitting light having a second wavelength (e.g., in the green range), the second wavelength being shorter than the first wavelength. Accordingly, the light from the first emitter 402 having the longer wavelength may penetrate deeper to reflect off the blood vessel 412, whereas the light from the second emitter 404 having the shorter wavelength may penetrate only to the shallower blood vessel 410.

As a result, the pulse signal measured by transmission of the light from the second emitter 404 to the sensor 406 may be delayed relative to the pulse signal measured by transmission of the light from the first emitter 402 to the sensor 406. This time lag may vary based on a respiratory rate as blood vessels and tissue such as 410 and 412 constrict and dilate, as discussed above.

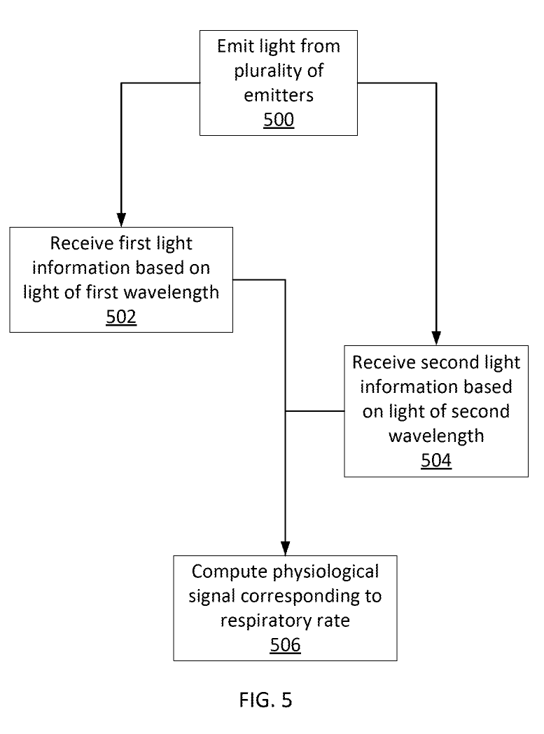

FIG. 5 illustrates a method of computing a physiological signal corresponding to respiratory rate of a user according to examples of the disclosure. Light may be emitted form a plurality of emitters (500). For example, light may be emitted from a first emitter at a first wavelength, and light may be emitted from a second emitter at a second wavelength. In some examples, the first wavelength (e.g., red or IR) may be longer than the second wavelength (e.g., green). First light information may be received based on light of the first wavelength (502), and second light information may be received based on light of the second wavelength (504). Then, based on the first and second light information, a physiological signal corresponding to the respiratory rate may be computed (506). For example, a time delay between the two signals may be determined as a function of time, and the fluctuating time delay may be taken as the respiratory rate signal. In some cases, the frequency of the fluctuating time delay may be determined as the respiratory rate.

The time delay as a function of time may be determined based on a cross-correlation of the first light information and the second light information. In some examples, the cross-correlation between the two signals may be computed in a sliding window over time, and the location of maximum cross-correlation may be taken as the average time delay for that particular window. For example, the cross-correlation may be calculated for a five-second window of the two signals, and the window may be advanced in one-second steps to determine the time delay as a function of time, although many other window lengths and interval steps may be possible.

The examples discussed above can be implemented in one or more Application Programming Interfaces (APIs). An API is an interface implemented by a program code component or hardware component (hereinafter “API-implementing component”) that allows a different program code component or hardware component (hereinafter “API-calling component”) to access and use one or more functions, methods, procedures, data structures, classes, and/or other services provided by the API-implementing component. An API can define one or more parameters that are passed between the API-calling component and the API-implementing component.

The above-described features can be implemented as part of an application program interface (API) that can allow it to be incorporated into different applications (e.g., spreadsheet apps) utilizing touch input as an input mechanism. An API can allow a developer of an API-calling component (which may be a third party developer) to leverage specified features, such as those described above, provided by an API-implementing component. There may be one API-calling component or there may be more than one such component. An API can be a source code interface that a computer system or program library provides in order to support requests for services from an application. An operating system (OS) can have multiple APIs to allow applications running on the OS to call one or more of those APIs, and a service (such as a program library) can have multiple APIs to allow an application that uses the service to call one or more of those APIs. An API can be specified in terms of a programming language that can be interpreted or compiled when an application is built.

In some examples, the API-implementing component may provide more than one API, each providing a different view of the functionality implemented by the API-implementing component, or with different aspects that access different aspects of the functionality implemented by the API-implementing component. For example, one API of an API-implementing component can provide a first set of functions and can be exposed to third party developers, and another API of the API-implementing component can be hidden (not exposed) and provide a subset of the first set of functions and also provide another set of functions, such as testing or debugging functions which are not in the first set of functions. In other examples the API-implementing component may itself call one or more other components via an underlying API and thus be both an API-calling component and an API-implementing component.

An API defines the language and parameters that API-calling components use when accessing and using specified features of the API-implementing component. For example, an API-calling component accesses the specified features of the API-implementing component through one or more API calls or invocations (embodied for example by function or method calls) exposed by the API and passes data and control information using parameters via the API calls or invocations. The API-implementing component may return a value through the API in response to an API call from an API-calling component. While the API defines the syntax and result of an API call (e.g., how to invoke the API call and what the API call does), the API may not reveal how the API call accomplishes the function specified by the API call. Various API calls are transferred via the one or more application programming interfaces between the calling (API-calling component) and an API-implementing component. Transferring the API calls may include issuing, initiating, invoking, calling, receiving, returning, or responding to the function calls or messages; in other words, transferring can describe actions by either of the API-calling component or the API-implementing component. The function calls or other invocations of the API may send or receive one or more parameters through a parameter list or other structure. A parameter can be a constant, key, data structure, object, object class, variable, data type, pointer, array, list or a pointer to a function or method or another way to reference a data or other item to be passed via the API.

Furthermore, data types or classes may be provided by the API and implemented by the API-implementing component. Thus, the API-calling component may declare variables, use pointers to, use or instantiate constant values of such types or classes by using definitions provided in the API.

Generally, an API can be used to access a service or data provided by the API-implementing component or to initiate performance of an operation or computation provided by the API-implementing component. By way of example, the API-implementing component and the API-calling component may each be any one of an operating system, a library, a device driver, an API, an application program, or other module (it should be understood that the API-implementing component and the API-calling component may be the same or different type of module from each other). API-implementing components may in some cases be embodied at least in part in firmware, microcode, or other hardware logic. In some examples, an API may allow a client program to use the services provided by a Software Development Kit (SDK) library. In other examples an application or other client program may use an API provided by an Application Framework. In these examples the application or client program may incorporate calls to functions or methods provided by the SDK and provided by the API or use data types or objects defined in the SDK and provided by the API. An Application Framework may in these examples provide a main event loop for a program that responds to various events defined by the Framework. The API allows the application to specify the events and the responses to the events using the Application Framework. In some implementations, an API call can report to an application the capabilities or state of a hardware device, including those related to aspects such as input capabilities and state, output capabilities and state, processing capability, power state, storage capacity and state, communications capability, etc., and the API may be implemented in part by firmware, microcode, or other low level logic that executes in part on the hardware component.

The API-calling component may be a local component (i.e., on the same data processing system as the API-implementing component) or a remote component (i.e., on a different data processing system from the API-implementing component) that communicates with the API-implementing component through the API over a network. It should be understood that an API-implementing component may also act as an API-calling component (i.e., it may make API calls to an API exposed by a different API-implementing component) and an API-calling component may also act as an API-implementing component by implementing an API that is exposed to a different API-calling component.

The API may allow multiple API-calling components written in different programming languages to communicate with the API-implementing component (thus the API may include features for translating calls and returns between the API-implementing component and the API-calling component); however the API may be implemented in terms of a specific programming language. An API-calling component can, in one example, call APIs from different providers such as a set of APIs from an OS provider and another set of APIs from a plug-in provider and another set of APIs from another provider (e.g. the provider of a software library) or creator of the another set of APIs.

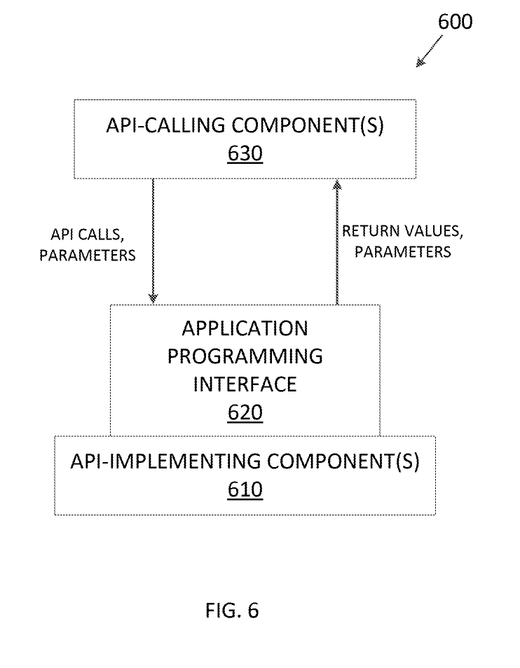

FIG. 6 is a block diagram illustrating an exemplary API architecture, which may be used in some examples of the disclosure. As shown in FIG. 4, the API architecture 600 includes the API-implementing component 610 (e.g., an operating system, a library, a device driver, an API, an application program, software or other module) that implements the API 620. The API 620 specifies one or more functions, methods, classes, objects, protocols, data structures, formats and/or other features of the API-implementing component that may be used by the API-calling component 630. The API 620 can specify at least one calling convention that specifies how a function in the API-implementing component receives parameters from the API-calling component and how the function returns a result to the API-calling component. The API-calling component 630 (e.g., an operating system, a library, a device driver, an API, an application program, software or other module), makes API calls through the API 620 to access and use the features of the API-implementing component 610 that are specified by the API 620. The API-implementing component 610 may return a value through the API 620 to the API-calling component 630 in response to an API call.

It will be appreciated that the API-implementing component 610 may include additional functions, methods, classes, data structures, and/or other features that are not specified through the API 620 and are not available to the API-calling component 630. It should be understood that the API-calling component 630 may be on the same system as the API-implementing component 610 or may be located remotely and accesses the API-implementing component 610 using the API 620 over a network. While FIG. 6 illustrates a single API-calling component 630 interacting with the API 620, it should be understood that other API-calling components, which may be written in different languages (or the same language) than the API-calling component 630, may use the API 620.

The API-implementing component 610, the API 620, and the API-calling component 630 may be stored in a non-transitory machine-readable storage medium, which includes any mechanism for storing information in a form readable by a machine (e.g., a computer or other data processing system). For example, a machine-readable medium includes magnetic disks, optical disks, random access memory; read only memory, flash memory devices, etc.

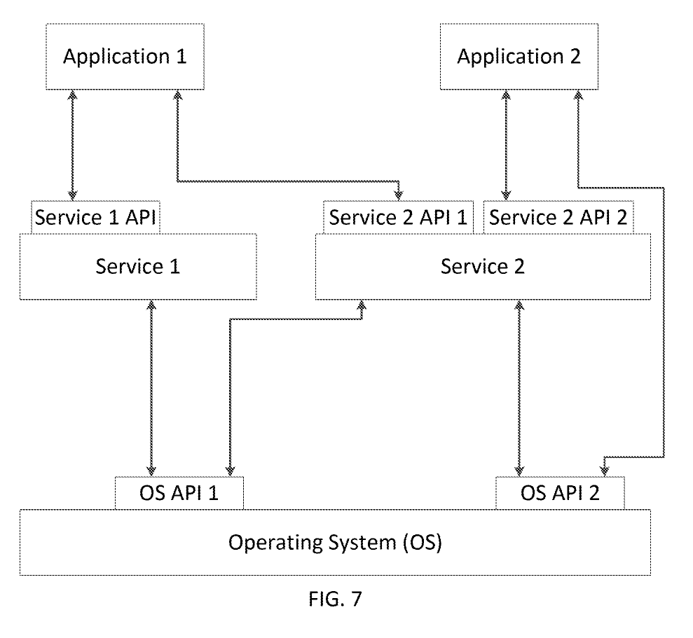

In the exemplary software stack shown in FIG. 7, applications can make calls to Services A or B using several Service APIs and to Operating System (OS) using several OS APIs. Services A and B can make calls to OS using several OS APIs.

Note that the Service 2 has two APIs, one of which (Service 2 API 1) receives calls from and returns values to Application 1 and the other (Service 2 API 2) receives calls from and returns values to Application 2. Service 1 (which can be, for example, a software library) makes calls to and receives returned values from OS API 1, and Service 2 (which can be, for example, a software library) makes calls to and receives returned values from both OS API 1 and OS API 2. Application 2 makes calls to and receives returned values from OS API 2.

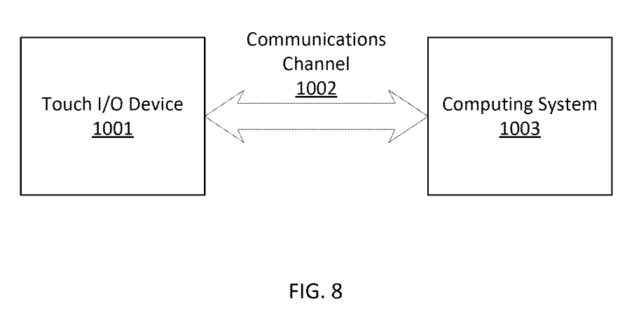

FIG. 8 is a block diagram illustrating exemplary interactions between the touch screen and the other components of the device. Described examples may include touch I/O device 1001 that can receive touch input for interacting with computing system 1003 via wired or wireless communication channel 1002. Touch I/O device 1001 may be used to provide user input to computing system 1003 in lieu of or in combination with other input devices such as a keyboard, mouse, etc. One or more touch I/O devices 1001 may be used for providing user input to computing system 1003. Touch I/O device 1001 may be an integral part of computing system 1003 (e.g., touch screen on a smartphone or a tablet PC) or may be separate from computing system 1003.

Touch I/O device 1001 may include a touch sensing panel which is wholly or partially transparent, semitransparent, non-transparent, opaque or any combination thereof. Touch I/O device 1001 may be embodied as a touch screen, touch pad, a touch screen functioning as a touch pad (e.g., a touch screen replacing the touchpad of a laptop), a touch screen or touchpad combined or incorporated with any other input device (e.g., a touch screen or touchpad disposed on a keyboard) or any multi-dimensional object having a touch sensing surface for receiving touch input.

In one example, touch I/O device 1001 embodied as a touch screen may include a transparent and/or semitransparent touch sensing panel partially or wholly positioned over at least a portion of a display. According to this example, touch I/O device 1001 functions to display graphical data transmitted from computing system 1003 (and/or another source) and also functions to receive user input. In other examples, touch I/O device 1001 may be embodied as an integrated touch screen where touch sensing components/devices are integral with display components/devices. In still other examples a touch screen may be used as a supplemental or additional display screen for displaying supplemental or the same graphical data as a primary display and to receive touch input.

Touch I/O device 1001 may be configured to detect the location of one or more touches or near touches on device 1001 based on capacitive, resistive, optical, acoustic, inductive, mechanical, chemical measurements, or any phenomena that can be measured with respect to the occurrences of the one or more touches or near touches in proximity to device 1001. Software, hardware, firmware or any combination thereof may be used to process the measurements of the detected touches to identify and track one or more gestures. A gesture may correspond to stationary or non-stationary, single or multiple, touches or near touches on touch I/O device 1001. A gesture may be performed by moving one or more fingers or other objects in a particular manner on touch I/O device 1001 such as tapping, pressing, rocking, scrubbing, twisting, changing orientation, pressing with varying pressure and the like at essentially the same time, contiguously, or consecutively. A gesture may be characterized by, but is not limited to a pinching, sliding, swiping, rotating, flexing, dragging, or tapping motion between or with any other finger or fingers. A single gesture may be performed with one or more hands, by one or more users, or any combination thereof.

Computing system 1003 may drive a display with graphical data to display a graphical user interface (GUI). The GUI may be configured to receive touch input via touch I/O device 1001. Embodied as a touch screen, touch I/O device 1001 may display the GUI. Alternatively, the GUI may be displayed on a display separate from touch I/O device 1001. The GUI may include graphical elements displayed at particular locations within the interface. Graphical elements may include but are not limited to a variety of displayed virtual input devices including virtual scroll wheels, a virtual keyboard, virtual knobs, virtual buttons, any virtual UI, and the like. A user may perform gestures at one or more particular locations on touch I/O device 1001 which may be associated with the graphical elements of the GUI. In other examples, the user may perform gestures at one or more locations that are independent of the locations of graphical elements of the GUI. Gestures performed on touch I/O device 1001 may directly or indirectly manipulate, control, modify, move, actuate, initiate or generally affect graphical elements such as cursors, icons, media files, lists, text, all or portions of images, or the like within the GUI. For instance, in the case of a touch screen, a user may directly interact with a graphical element by performing a gesture over the graphical element on the touch screen. Alternatively, a touch pad generally provides indirect interaction. Gestures may also affect non-displayed GUI elements (e.g., causing user interfaces to appear) or may affect other actions within computing system 1003 (e.g., affect a state or mode of a GUI, application, or operating system). Gestures may or may not be performed on touch I/O device 1001 in conjunction with a displayed cursor. For instance, in the case in which gestures are performed on a touchpad, a cursor (or pointer) may be displayed on a display screen or touch screen and the cursor may be controlled via touch input on the touchpad to interact with graphical objects on the display screen. In other examples in which gestures are performed directly on a touch screen, a user may interact directly with objects on the touch screen, with or without a cursor or pointer being displayed on the touch screen.

Feedback may be provided to the user via communication channel 1002 in response to or based on the touch or near touches on touch I/O device 1001. Feedback may be transmitted optically, mechanically, electrically, olfactory, acoustically, or the like or any combination thereof and in a variable or non-variable manner.

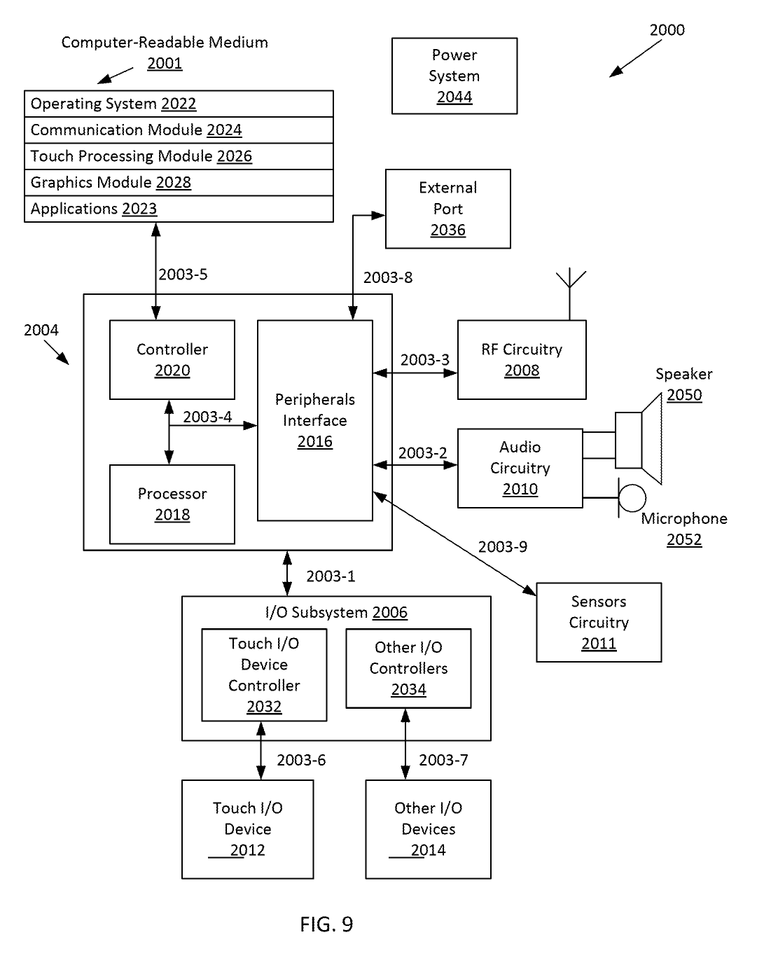

Attention is now directed towards examples of a system architecture that may be embodied within any portable or non-portable device including but not limited to a communication device (e.g. mobile phone, smart phone), a multi-media device (e.g., MP3 player, TV, radio), a portable or handheld computer (e.g., tablet, netbook, laptop), a desktop computer, an All-In-One desktop, a peripheral device, or any other system or device adaptable to the inclusion of system architecture 2000, including combinations of two or more of these types of devices. FIG. 9 is a block diagram of one example of system 2000 that generally includes one or more computer-readable mediums 2001, processing system 2004, I/O subsystem 2006, radio frequency (RF) circuitry 2008, audio circuitry 2010, and sensors circuitry 2011. These components may be coupled by one or more communication buses or signal lines 2003.

It should be apparent that the architecture shown in FIG. 9 is only one example architecture of system 2000, and that system 2000 could have more or fewer components than shown, or a different configuration of components. The various components shown in FIG. 9 can be implemented in hardware, software, firmware or any combination thereof, including one or more signal processing and/or application specific integrated circuits.

RF circuitry 2008 can be used to send and receive information over a wireless link or network to one or more other devices and includes well-known circuitry for performing this function. RF circuitry 2008 and audio circuitry 2010 can be coupled to processing system 2004 via peripherals interface 2016. Interface 2016 can include various known components for establishing and maintaining communication between peripherals and processing system 2004. Audio circuitry 2010 can be coupled to audio speaker 2050 and microphone 2052 and can include known circuitry for processing voice signals received from interface 2016 to enable a user to communicate in real-time with other users. In some examples, audio circuitry 2010 can include a headphone jack (not shown). Sensors circuitry 2011 can be coupled to various sensors including, but not limited to, one or more Light Emitting Diodes (LEDs) or other light emitters, one or more photodiodes or other light sensors, one or more photothermal sensors, a magnetometer, an accelerometer, a gyroscope, a barometer, a compass, a proximity sensor, a camera, an ambient light sensor, a thermometer, a GPS sensor, and various system sensors which can sense remaining battery life, power consumption, processor speed, CPU load, and the like.

Peripherals interface 2016 can couple the input and output peripherals of the system to processor 2018 and computer-readable medium 2001. One or more processors 2018 communicate with one or more computer-readable mediums 2001 via controller 2020. Computer-readable medium 2001 can be any device or medium that can store code and/or data for use by one or more processors 2018. In some examples, medium 2001 can be a non-transitory computer-readable storage medium. Medium 2001 can include a memory hierarchy, including but not limited to cache, main memory and secondary memory. The memory hierarchy can be implemented using any combination of RAM (e.g., SRAM, DRAM, DDRAM), ROM, FLASH, magnetic and/or optical storage devices, such as disk drives, magnetic tape, CDs (compact disks) and DVDs (digital video discs). Medium 2001 may also include a transmission medium for carrying information-bearing signals indicative of computer instructions or data (with or without a carrier wave upon which the signals are modulated). For example, the transmission medium may include a communications network, including but not limited to the Internet (also referred to as the World Wide Web), intranet(s), Local Area Networks (LANs), Wide Local Area Networks (WLANs), Storage Area Networks (SANs), Metropolitan Area Networks (MAN) and the like.

One or more processors 2018 can run various software components stored in medium 2001 to perform various functions for system 2000. In some examples, the software components can include operating system 2022, communication module (or set of instructions) 2024, touch processing module (or set of instructions) 2026, graphics module (or set of instructions) 2028, and one or more applications (or set of instructions) 2030. Each of these modules and above noted applications can correspond to a set of instructions for performing one or more functions described above and the methods described in this application (e.g., the computer-implemented methods and other information processing methods described herein). These modules (i.e., sets of instructions) need not be implemented as separate software programs, procedures or modules, and thus various subsets of these modules may be combined or otherwise re-arranged in various examples. In some examples, medium 2001 may store a subset of the modules and data structures identified above. Furthermore, medium 2001 may store additional modules and data structures not described above.

Operating system 2022 can include various procedures, sets of instructions, software components and/or drivers for controlling and managing general system tasks (e.g., memory management, storage device control, power management, etc.) and facilitates communication between various hardware and software components.

Communication module 2024 can facilitate communication with other devices over one or more external ports 2036 or via RF circuitry 2008 and can include various software components for handling data received from RF circuitry 2008 and/or external port 2036.

Graphics module 2028 can include various known software components for rendering, animating and displaying graphical objects on a display surface. In examples in which touch I/O device 2012 is a touch sensing display (e.g., touch screen), graphics module 2028 can include components for rendering, displaying, and animating objects on the touch sensing display.

One or more applications 2030 can include any applications installed on system 2000, including without limitation, a browser, address book, contact list, email, instant messaging, word processing, keyboard emulation, widgets, JAVA-enabled applications, encryption, digital rights management, voice recognition, voice replication, location determination capability (such as that provided by the global positioning system (GPS)), a music player, etc.

Touch processing module 2026 can include various software components for performing various tasks associated with touch I/O device 2012 including but not limited to receiving and processing touch input received from I/O device 2012 via touch I/O device controller 2032.

I/O subsystem 2006 can be coupled to touch I/O device 2012 and one or more other I/O devices 2014 for controlling or performing various functions. Touch I/O device 2012 can communicate with processing system 2004 via touch I/O device controller 2032, which can include various components for processing user touch input (e.g., scanning hardware). One or more other input controllers 2034 can receive/send electrical signals from/to other I/O devices 2014. Other I/O devices 2014 may include physical buttons, dials, slider switches, sticks, keyboards, touch pads, additional display screens, or any combination thereof.

If embodied as a touch screen, touch I/O device 2012 can display visual output to the user in a GUI. The visual output may include text, graphics, video, and any combination thereof. Some or all of the visual output may correspond to user-interface objects. Touch I/O device 2012 can form a touch sensing surface that accepts touch input from the user. Touch I/O device 2012 and touch screen controller 2032 (along with any associated modules and/or sets of instructions in medium 2001) can detect and track touches or near touches (and any movement or release of the touch) on touch I/O device 2012 and can convert the detected touch input into interaction with graphical objects, such as one or more user-interface objects. In the case in which device 2012 is embodied as a touch screen, the user can directly interact with graphical objects that are displayed on the touch screen. Alternatively, in the case in which device 2012 is embodied as a touch device other than a touch screen (e.g., a touch pad), the user may indirectly interact with graphical objects that are displayed on a separate display screen embodied as I/O device 2014.

Touch I/O device 2012 may be analogous to the multi-touch sensing surface described in the following U.S. Pat. No. 6,323,846 (Westerman et al.), U.S. Pat. No. 6,570,557 (Westerman et al.), and/or U.S. Pat. No. 6,677,932 (Westerman), and/or U.S. Patent Publication 2002/0015024A1, each of which is hereby incorporated by reference.

In examples for which touch I/O device 2012 is a touch screen, the touch screen may use LCD (liquid crystal display) technology, LPD (light emitting polymer display) technology, OLED (organic LED), or OEL (organic electro luminescence), although other display technologies may be used in other examples.

Feedback may be provided by touch I/O device 2012 based on the user's touch input as well as a state or states of what is being displayed and/or of the computing system. Feedback may be transmitted optically (e.g., light signal or displayed image), mechanically (e.g., haptic feedback, touch feedback, force feedback, or the like), electrically (e.g., electrical stimulation), olfactory, acoustically (e.g., beep or the like), or the like or any combination thereof and in a variable or non-variable manner.

System 2000 can also include power system 2044 for powering the various hardware components and may include a power management system, one or more power sources, a recharging system, a power failure detection circuit, a power converter or inverter, a power status indicator and any other components typically associated with the generation, management and distribution of power in portable devices.

In some examples, peripherals interface 2016, one or more processors 2018, and memory controller 2020 may be implemented on a single chip, such as processing system 2004. In some other examples, they may be implemented on separate chips.

Examples of the disclosure can be advantageous in allowing for an electronic device to obtain a respiratory rate signal from a PPG signal, making for a more accurate reading of respiratory rate without directly monitoring breathing.

In some examples, a method of an electronic device including a plurality of light emitters may be disclosed. The method may include: emitting light from the plurality of light emitters; receiving first light information based on light emitted at a first wavelength from a first light emitter; receiving second light information based on light emitted at a second wavelength from a second light emitter; and based on a cross-correlation of the first and second light information, computing a physiological signal corresponding to a respiratory rate of a user of the electronic device. Additionally or alternatively to one or more of the above examples, the method may further comprise: determining the cross-correlation of the first and second light information in a sliding window; and determining an average time delay in the sliding window based on the location of maximum cross-correlation, wherein the respiratory rate may be determined based on the frequency of fluctuating time delay. Additionally or alternatively to one or more of the above examples, the first wavelength may be longer than the second wavelength, the second light information may have a time delay with respect to the first light information, and the physiological signal corresponding to the respiratory rate may be determined based on an estimate of the time delay as a function of time. Additionally or alternatively to one or more of the above examples, receiving the first light information may include receiving light of the first wavelength at a light sensor, and receiving the second light information may include receiving light of the second wavelength at the light sensor. Additionally or alternatively to one or more of the above examples, computing the physiological signal corresponding to the respiratory rate may include determining a frequency of a fluctuating time delay between the first and second light information. Additionally or alternatively to one or more of the above examples, the method may further comprise determining a heart rate signal based on the first and second light information. Additionally or alternatively to one or more of the above examples, the first wavelength may be one of infrared and red, and the second wavelength may be green.

In some examples, a non-transitory computer readable storage medium may be disclosed. The computer readable medium may contain instructions that, when executed, perform a method of an electronic device including a plurality of light emitters may be disclosed. The method may include: emitting light from the plurality of light emitters; receiving first light information based on light emitted at a first wavelength from a first light emitter; receiving second light information based on light emitted at a second wavelength from a second light emitter; and based on a cross-correlation of the first and second light information, computing a physiological signal corresponding to a respiratory rate of a user of the electronic device. Additionally or alternatively to one or more of the above examples, the method may further comprise: determining the cross-correlation of the first and second light information in a sliding window; and determining an average time delay in the sliding window based on the location of maximum cross-correlation, wherein the respiratory rate may be determined based on the frequency of fluctuating time delay. Additionally or alternatively to one or more of the above examples, the first wavelength may be longer than the second wavelength, the second light information may have a time delay with respect to the first light information, and the physiological signal corresponding to the respiratory rate may be determined based on an estimate of the time delay as a function of time. Additionally or alternatively to one or more of the above examples, receiving the first light information may include receiving light of the first wavelength at a light sensor, and receiving the second light information may include receiving light of the second wavelength at the light sensor. Additionally or alternatively to one or more of the above examples, computing the physiological signal corresponding to the respiratory rate may include determining a frequency of a fluctuating time delay between the first and second light information. Additionally or alternatively to one or more of the above examples, the method may further comprise determining a heart rate signal based on the first and second light information. Additionally or alternatively to one or more of the above examples, the first wavelength may be one of infrared and red, and the second wavelength may be green.

In some examples, an electronic device may be disclosed. The electronic device may include: a processor to execute instructions; a plurality of light emitters; and a memory coupled with the processor to store instructions, which when executed by the processor, may cause the processor to perform operations to generate an application programming interface (API) that allows an API-calling component to perform a method. The method may include: emitting light from the plurality of light emitters; receiving first light information based on light emitted at a first wavelength from a first light emitter; receiving second light information based on light emitted at a second wavelength from a second light emitter; and based on a cross-correlation of the first and second light information, computing a physiological signal corresponding to a respiratory rate of a user of the electronic device. Additionally or alternatively to one or more of the above examples, the method may further comprise: determining the cross-correlation of the first and second light information in a sliding window; and determining an average time delay in the sliding window based on the position of maximum cross-correlation, wherein the respiratory rate may be determined based on the frequency of fluctuating time delay. Additionally or alternatively to one or more of the above examples, the first wavelength may be longer than the second wavelength, the first light information may have a time delay with respect to the second light information, and the physiological signal corresponding to the respiratory rate may be determined based on an estimate of the time delay as a function of time. Additionally or alternatively to one or more of the above examples, receiving the first light information may include receiving light of the first wavelength at a light sensor, and receiving the second light information may include receiving light of the second wavelength at the light sensor. Additionally or alternatively to one or more of the above examples, computing the physiological signal corresponding to the respiratory rate may include determining a frequency of a fluctuating time delay between the first and second light information. Additionally or alternatively to one or more of the above examples, the method may further comprise determining a heart rate signal based on the first and second light information. Additionally or alternatively to one or more of the above examples, the first wavelength may be one of infrared and red, and the second wavelength may be green.

Although the disclosed examples have been fully described with reference to the accompanying drawings, it is to be noted that various changes and modifications will become apparent to those skilled in the art. Such changes and modifications are to be understood as being included within the scope of the disclosed examples as defined by the appended claims.

1. A method of an electronic device including a plurality of light emitters, the method comprising:
emitting light from the plurality of light emitters;
receiving first light information based on light emitted at a first wavelength from a first light emitter;
receiving second light information based on light emitted at a second wavelength from a second light emitter; and
based on a cross-correlation of the first and second light information, computing a physiological signal corresponding to a respiratory rate of a user of the electronic device.

2. The method of claim 1, the method further comprising:
determining the cross-correlation of the first and second light information in a sliding window; and
determining an average time delay in the sliding window based on a position of maximum cross-correlation, wherein the respiratory rate is determined based on a frequency of fluctuating time delay.

3. The method of claim 1, the method further comprising:
determining the cross-correlation of the first and second light information in a sliding window; and
determining an average time delay in the sliding window based on a position of maximum cross-correlation, wherein the respiratory rate is determined based on the average time delay.

4. The method of claim 1, wherein the first wavelength is longer than the second wavelength, the second light information has a time delay with respect to the first light information, and the physiological signal corresponding to the respiratory rate is determined based on an estimate of the time delay as a function of time.

5. The method of claim 1, wherein receiving the first light information includes receiving light of the first wavelength at a light sensor, and receiving the second light information includes receiving light of the second wavelength at the light sensor.

6. The method of claim 1, wherein computing the physiological signal corresponding to the respiratory rate includes determining a frequency of a fluctuating time delay between the first and second light information.

7. The method of claim 1, the method further comprising determining a heart rate signal based on the first and second light information.

8. The method of claim 1, wherein the first wavelength is one of infrared and red, and the second wavelength is green.

9. A non-transitory computer readable medium, the computer readable medium containing instructions that, when executed, perform a method of an electronic device including a plurality of light emitters, the method comprising:
emitting light from the plurality of light emitters;
receiving first light information based on light emitted at a first wavelength from a first light emitter;
receiving second light information based on light emitted at a second wavelength from a second light emitter; and
based on a cross-correlation of the first and second light information, computing a physiological signal corresponding to a respiratory rate of a user of the electronic device.

10. The non-transitory computer readable medium of claim 9, the method further comprising:
determining the cross-correlation of the first and second light information in a sliding window; and
determining an average time delay in the sliding window based on a position of maximum cross-correlation, wherein the respiratory rate is determined based on a frequency of fluctuating time delay.

11. The non-transitory computer readable medium of claim 9, the method further comprising:
determining the cross-correlation of the first and second light information in a sliding window; and
determining an average time delay in the sliding window based on the position of maximum cross-correlation, wherein the respiratory rate is determined based on the average time delay.

12. The non-transitory computer readable medium of claim 9, wherein the first wavelength is longer than the second wavelength, the second light information has a time delay with respect to the first light information, and the physiological signal corresponding to the respiratory rate is determined based on an estimate of the time delay as a function of time.

13. The non-transitory computer readable medium of claim 9, wherein receiving the first light information includes receiving light of the first wavelength at a light sensor, and receiving the second light information includes receiving light of the second wavelength at the light sensor.

14. The non-transitory computer readable medium of claim 9, wherein computing the physiological signal corresponding to the respiratory rate includes determining a frequency of a fluctuating time delay between the first and second light information.

15. The non-transitory computer readable medium of claim 9, the method further comprising determining a heart rate signal based on the first and second light information.

16. The non-transitory computer readable medium of claim 9, wherein the first wavelength is one of infrared and red, and the second wavelength is green.

17. An electronic device, comprising:
a first light emitter configured to emit a first light, the first light including a first wavelength and a first light information;
a second light emitter configured to emit a second light, the second light including a second wavelength and a second light information;
a detector configured to receive the first light and the second light; and
a processor configured to:
receive first light information;
receive second light information; and
based on a cross-correlation of the first and second light information, compute a physiological signal corresponding to a respiratory rate of a user of the electronic device.

18. The electronic device of claim 17, wherein the processor is further configured to:
determine the cross-correlation of the first and second light information in a sliding window; and
determine an average time delay in the sliding window based on the position of maximum cross-correlation, wherein the respiratory rate is determined based on the frequency of fluctuating time delay.

19. The electronic device of claim 17, wherein the processor is further configured to:
determine the cross-correlation of the first and second light information in a sliding window; and
determine an average time delay in the sliding window based on the position of maximum cross-correlation, wherein the respiratory rate is determined based on the average time delay.

20. The electronic device of claim 17, wherein the first wavelength is longer than the second wavelength, the second light information has a time delay with respect to the first light information, and the physiological signal corresponding to the respiratory rate is determined based on an estimate of the time delay as a function of time.

21. The electronic device of claim 17, wherein receiving the first light information includes receiving light of the first wavelength at a light sensor, and receiving the second light information includes receiving light of the second wavelength at the light sensor.

22. The electronic device of claim 17, wherein computing the physiological signal corresponding to the respiratory rate includes determining a frequency of a fluctuating time delay between the first and second light information.

23. The electronic device of claim 17, wherein the processor is further configured to determine a heart rate signal based on the first and second light information.

24. The electronic device of claim 17, wherein the first wavelength is one of infrared and red, and the second wavelength is green.
